(19) United States
(12) Patent Application Publication
(10) Pub. No.: US 2011/0166152 A1
(43) Pub. Date: Jul. 7, 2011

(54) HETEROAROMATIC COMPOUNDS AS INHIBITORS OF STEAROYL-COENZYME A DELTA-9 DESATURASE

(76) Inventors: Jean-Philippe Leclerc, Laval (CA); Chun Sing Li, Dollard-des-Ormeaux (CA); Yeeman K. Ramtohul, Pierrefonds (CA)

(21) Appl. No.: 13/119,467

(22) PCT Filed: Sep. 30, 2009
(86) PCT No.: PCT/CA09/01387

§ 371 Date: Mar. 17, 2011

Related U.S. Application Data
(60) Provisional application No. 61/195,003, filed on Oct. 2, 2008.

(51) Int. Cl. A61K 31/4985; C07D 277/62; C07D 487/04; C07D 471/04; A61K 31/426; A61K 31/437; A61P 3/10; A61P 9/10

(52) U.S. Cl. 514/249; 548/180; 544/350; 546/121; 514/367; 514/303

(57) ABSTRACT

Heteroaromatic compounds of structural formula (I) are inhibitors of stearoyl-coenzyme A delta-9 desaturase (SCD). The compounds of the present invention are useful for the prevention and treatment of conditions related to abnormal lipid synthesis and metabolism, including cardiovascular disease, such as atherosclerosis; obesity; Type 2 diabetes; insulin resistance; hyperglycemia; Metabolic Syndrome; neurological disease; cancer; and liver steatosis.

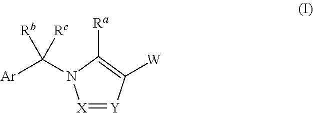

### FIELD OF THE INVENTION

The present invention relates to heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease, such as atherosclerosis; obesity; diabetes; neurological disease; metabolic syndrome; insulin resistance; cancer; and hepatic steatosis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). Elevated SCD activity in adipose tissue is closely coupled to the development of insulin resistance (Sjogren, et al., Diabetologia, 51(2): 328-35 (2007)). The postprandial de novo lipogenesis is significantly elevated in obese subjects (Marques-Lopes, et al., American Journal of Clinical Nutrition, 73: 252-261 (2001)). Knockout of the SCD gene ameliorates Metabolic Syndrome by reducing plasma triglycerides, reducing weight gain, increasing insulin sensitivity, and reduces hepatic lipid accumulation (MacDonald, et al., Journal of Lipid Research, 49(1): 217-29 (2007)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)). RNA interference of SCD-1 reduces human tumor cell survival (Morgan-Lappe, et al., Cancer Research, 67(9): 4390-4398 (2007)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications WO 2005/011653, WO 2005/011654, WO 2005/011656, WO 2005/011656, WO 2005/011657, WO 2006/014168, WO 2006/034279, WO 2006/034312, WO 2006/034315, WO 2006/034338, WO 2006/034341, WO 2006/034440, WO 2006/034441, WO 2006/034446, WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; WO 2007/136746; and WO 2008/074835, all assigned to Xenon Pharmaceuticals, Inc.

A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/056846 (24 May 2007); WO 2007/071023 (28 Jun. 2007); WO 2007/134457 (29 Nov. 2007); WO 2007/143823 (21 Dec. 2007); WO 2007/143824 Dec. 2007); WO 2008/017161 (14 Feb. 2008); WO 2008/046226 (24 Apr. 2008); WO 2008/064474 (5 Jun. 2008); and US 2008/0182838 (31 Jul. 2008).

WO 2008/003753 (assigned to Novartis) discloses a series of pyrazolo[1,5-a]pyrimidine analogs as SCD inhibitors; WO 2007/143597 and WO 2008/024390 (assigned to Novartis and Xenon Pharmaceuticals) disclose heterocyclic derivatives as SCD inhibitors; and WO 2008/096746 (assigned to Takeda Pharmaceutical) disclose Spiro compounds as SCD inhibitors.

Small molecule SCD inhibitors have also been described by (a) G. Liu, et al., “Discovery of Potent, Selective, Orally Bioavailable SCD1 Inhibitors,” in J. Med. Chem., 50: 3086-3100 (2007); (b) H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007); and (c) Z. Xin, et al., “Discovery of piperidine-aryl urea-based stearoyl-CoA desaturase 1 inhibitors,” Bioorg. Med. Chem. Lett., 18: 4298-4302 (2008).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobrzyn and J. M. Ntambi, in “Stearoyl-CoA desaturase as a new drug target for obesity treatment,” Obesity Reviews, 6: 169-174 (2005).

### SUMMARY OF THE INVENTION

The present invention relates to heteroaromatic compounds of structural formula I:

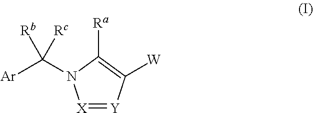

These heteroaromatic compounds are effective as inhibitors of SCD. They are therefore useful for the treatment, control or prevention of disorders responsive to the inhibition of SCD, such as diabetes, insulin resistance, lipid disorders, obesity, atherosclerosis, metabolic syndrome, and cancer.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, metabolic syndrome, and cancer by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of insulin resistance by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for the treatment, control, or prevention of metabolic syndrome by administering the compounds of the present invention in combination with a therapeutically effective amount of another agent known to be useful to treat the condition.

The present invention also relates to methods for the treatment, control, or prevention of cancer by administering the compounds of the present invention in combination with a therapeutically effective amount of another agent known to be useful to treat the condition.

### DETAILED DESCRIPTION OF THE INVENTION

The present invention is concerned with heteroaromatic compounds useful as inhibitors of SCD. Compounds of the present invention are described by structural formula I:

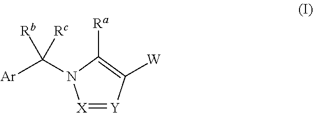

and pharmaceutically acceptable salts thereof; wherein
X and Y are each independently CH or N;
W is heteroaryl selected from the group consisting of:

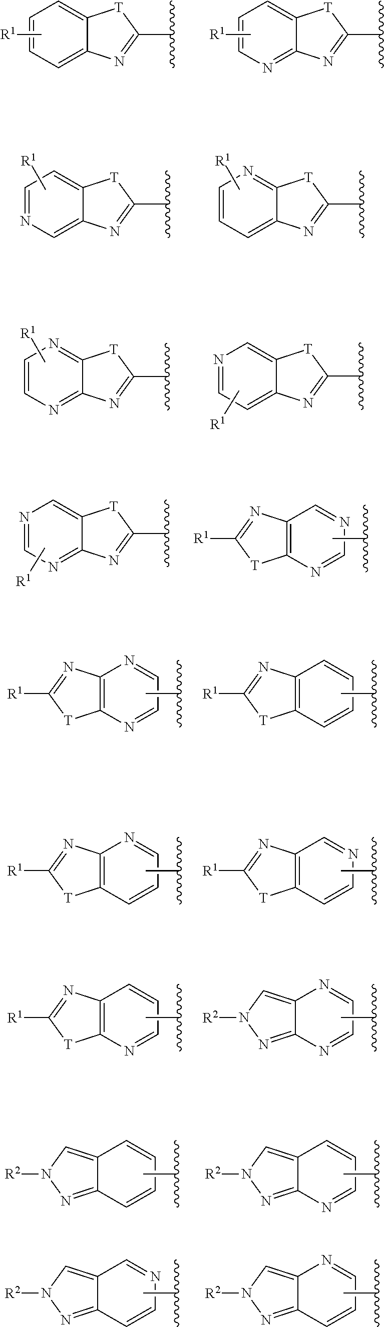

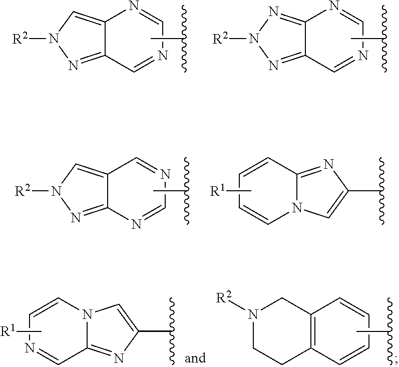

wherein W is further optionally substituted with one to two substituents independently selected from R⁴;
R¹ is selected from the group consisting of:

—(CH₂)ₚCO₂H,

—(CH₂)ₚCO₂C₁₋₄ alkyl,

—Z(CH₂)ₘCO₂H,

—Z(CH₂)ₘCO₂C₁₋₄ alkyl,

—(CH₂)ₙOR⁶,

—(CH₂)ₙ—CONR⁶R⁷,

—(CH₂)ₙ—OCONR⁶R⁷,

—(CH₂)ₙ—SO₂NR⁶R⁷,

—(CH₂)ₙ—SO₂R⁸,

—(CH₂)ₙ—NR⁹SO₂R⁸,

—(CH₂)ₙ—NR⁹CONR⁶R⁷,

—(CH₂)ₙ—NR⁹COR⁹, and

—(CH₂)ₙ—NR⁹CO₂R⁸;

R² is —(CH₂)ₘCO₂H or —(CH₂)ₘCO₂C₁₋₃ alkyl;
each m is independently an integer from 1 to 3;
each n is independently an integer from 0 to 3;
each p is independently an integer from 0 to 3;

### T is O, S, or NR⁵;

### Z is O, S, or NR⁵;

each R⁴ is independently selected from the group consisting of:

hydrogen,

halogen,

cyano,

C₁₋₄ alkyl, optionally substituted with one to five fluorines,

C₁₋₄ alkoxy, optionally substituted with one to five fluorines,

C₁₋₄ alkylthio, optionally substituted with one to five fluorines,

C₁₋₄ alkylsulfonyl,

carboxy,

C₁₋₄ alkyloxycarbonyl, and

C₁₋₄ alkylcarbonyl;

R⁵ is hydrogen or C₁₋₄ alkyl wherein alkyl is optionally substituted with one to five fluorines;
R⁶ and R⁷ are each independently selected from the group consisting of

hydrogen,

(CH₂)ₙ-phenyl,

(CH₂)ₙ—C₃₋₆ cycloalkyl, and

C₁₋₆ alkyl, wherein alkyl is optionally substituted with one to five substituents independently selected from fluorine and hydroxy and wherein phenyl and cycloalkyl are optionally substituted with one to five substituents independently selected from halogen, hydroxy, C₁₋₆ alkyl, and C₁₋₆ alkoxy, wherein alkyl and alkoxy are optionally substituted with one to five fluorines;
or R⁶ and R⁷ together with the nitrogen atom to which they are attached form a heterocyclic ring selected from azetidine, pyrrolidine, piperidine, piperazine, and morpholine wherein said heterocyclic ring is optionally substituted with one to three substituents independently selected from halogen, hydroxy, C₁₋₆ alkyl, and C₁₋₆ alkoxy, wherein alkyl and alkoxy are optionally substituted with one to five fluorines;
   each R⁸ is independently C₁₋₆ alkyl, wherein alkyl is optionally substituted with one to five substituents independently selected from fluorine and hydroxyl;
   R⁹ is hydrogen or R⁸;
   Ar is phenyl or pyridyl each of which is optionally substituted with one to five substituents independently selected from the group consisting of:

halogen,

C₁₋₆ alkyl optionally substituted with one to five fluorines,

C₂₋₆ alkenyl,

C₂₋₆ alkynyl,

C₁₋₆ alkylthio, optionally substituted with one to five fluorines,

C₁₋₆ alkoxy, optionally substituted with one to five fluorines, and

C₃₋₆ cycloalkyl;

Ra is hydrogen or C₁₋₄ alkyl wherein alkyl is optionally substituted with one to five fluorines; and
R^{b} and R^{c} are each independently hydrogen, fluorine, or C₁₋₄ alkyl wherein alkyl is optionally substituted with one to five fluorines;
or R^{b} and R^{c} are taken together to form a 3- to 6-membered saturated carbocyclic ring optionally containing a heteroatom selected from the group consisting of O, S, and N.

In one embodiment of the compounds of the present invention, X and Y are both N. In a class of this embodiment, R^{a}, R^{b}, and R^{c} are each hydrogen, and Ar is phenyl substituted with one to five substituents independently selected from the group consisting of halogen and C₁₋₄ alkyl.

In a second embodiment of the compounds of the present invention, W is heteroaryl selected from the group consisting of:

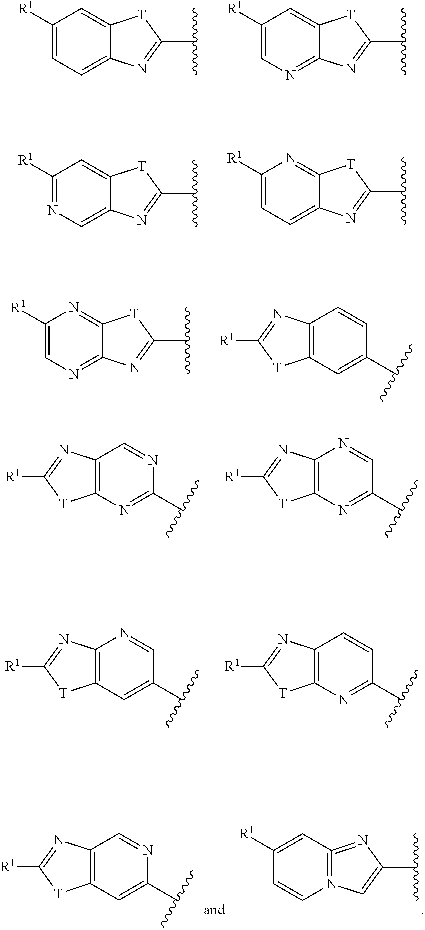

In a class of this embodiment, T is O or S. In a subclass of this class, T is S.

In a second class of this embodiment, X and Y are both N. In a subclass of this class, R^{a}, R^{b}, and R^{c} are each hydrogen, and Ar is phenyl substituted with one to five substituents independently selected from the group consisting of halogen and C₁₋₄ alkyl.

In a third embodiment of the compounds of the present invention, W is heteroaryl selected from the group consisting of:

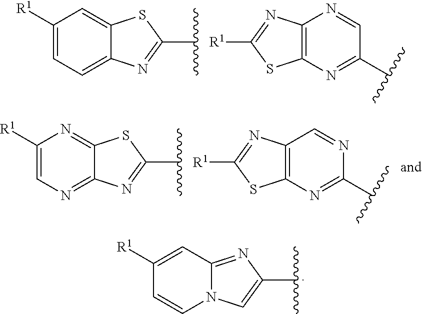

In a class of this embodiment, X and Y are both N. In a subclass of this class, R^{a}, R^{b}, and R^{c} are each hydrogen, and Ar is phenyl substituted with one to five substituents independently selected from the group consisting of halogen and C₁₋₄ alkyl.

Illustrative, but nonlimiting examples, of compounds of the present invention that are useful as inhibitors of SCD are the following:

| Example | IC₅₀ hSCD-1 |
|---|---|
| 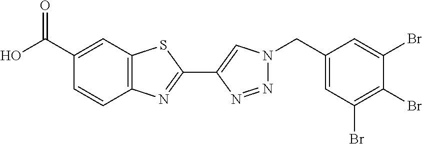 | 14 nM |
| 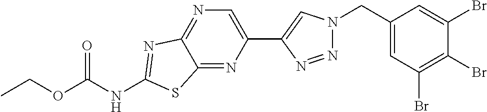 | 16 nM |
| 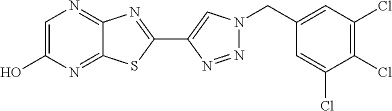 | 48 nM |
| 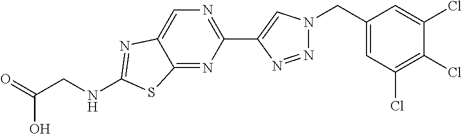 | 31 nM |
| 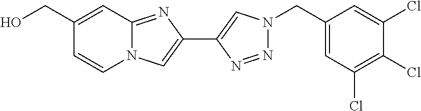 | 39 nM |

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. When no number of carbon atoms is specified, C₁₋₆ is intended.

The term “alkenyl” shall mean straight or branched-chain alkenes having the specified number of carbon atoms. Examples of alkenyl include vinyl, 1-propenyl, 1-butenyl, 2-butenyl, and the like.

The term “alkynyl” refers to straight or branched-chain alkynes having the specified number of carbon atoms. Examples of alkynyl include ethynyl, propynyl, butynyl, pentynyl, and the like.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Cycloalkyl” means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

As defined herein, a condition or disease mediated by high or abnormal SCD enzyme activity is defined as any disease or condition in which the activity of SCD is elevated and/or where inhibition of SCD can be demonstrated to bring about symptomatic improvements for the individual so treated. As defined herein, a condition or disease mediated by high or abnormal SCD enzyme activity includes, but is not limited to cardiovascular disease, dyslipidemias, (including but not limiting to disorders of serum levels of triglycerides, hypertriglyceridemia, VLDL, HDL, LDL, cholesterol, and total cholesterol, hypercholesterolemia, as well as cholesterol disorders), familial combined hyperlipidemia, coronary artery disease, atherosclerosis, heart disease, cerebrovascular disease (including but not limited to stroke, ischemic stroke, and transient ischemic attack), peripheral vascular disease, and ischemic retinopathy.

A condition or disease mediated by high or abnormal SCD enzyme activity also includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders (including but not limited to obesity, overweight, cacahexia, and anorexia), weight loss, body mass index and leptin-related diseases.

A condition or disease mediated by high or abnormal SCD enzyme activity also includes fatty liver, hepatic steatosis, hepatitis, non-alcoholic hepatitis, non-alcoholic steatohepatitis, alcoholic hepatitis, acute fatty liver, fatty liver of pregnancy, drug-induced hepatitis, erythrohepatic protporphyria, iron overload disorders, hereditary hemochromatosis, hepatic fibrosis, hepatic cirrhosis, hepatoma and conditions related thereto.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequalae in a mammalian patient in need of such treatment comprising administering to said patient a compound in accordance with structural formula I in an amount that is effective to treat metabolic syndrome and its sequalae. The sequalae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequalae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequalae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, and other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequalae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD Enzyme Activity Assay:

The potency of compounds of formula I against the stearoyl-CoA desaturase was determined by measuring the conversion of radiolabeled stearoyl-CoA to oleoyl-CoA using rat liver microsome or human SCD1 following previously published procedures with some modifications (Joshi, et al., J. Lipid Res., 18: 32-36 (1977); Talamo, et al., Anal. Biochem, 29: 300-304 (1969)). Liver microsome was prepared from male Wistar or Sprague Dawley rats on a high carbohydrate diet for 3 days (LabDiet #5803, Purina). The livers were homogenized (1:10 w/v) in a buffer containing 250 mM sucrose, 1 mM EDTA, 5 mM DTT and 50 mM Tris-HCl (pH 7.5). After a 100,000×g centrifugation for 60 min, the liver microsome pellet was suspended in a buffer containing 100 mM sodium phosphate, 20% glycerol, 2 mM DTT, and stored at −78° C. Human SCD1 desaturase system was reconstituted using human SCD1 from a baculovirus/Sf9 expression system, cytochrome B5 and cytochrome B5 reductase. Typically, test compound in 2 μL DMSO was incubated for 15 min at room temperature with 180 μL of the SCD enzyme in a buffer containing 100 mM Tris-HCl (pH 7.5), ATP (5 mM), Coenzyme-A (0.1 mM), Triton X-100 (0.5 mM) and NADH (2 mM). The reaction was initiated by the addition of 20 μL of [³H]-stearoyl-CoA (final concentration=2 μM, radioactivity concentration=1 μCi/mL). After 10 min, the reaction mixture (80 μL) was mixed with a calcium chloride/charcoal aqueous suspension (100 μL charcoal (10% w/v) plus 25 μL CaCl₂ (2N). After centrifugation to precipitate the radioactive fatty acid species, tritiated water released from 9,10-[³H]-stearoyl-CoA by the SCD enzyme was quantified on a scintillation counter.

### II. Whole Cell-Based SCD (Delta-9), Delta-5 and Delta-6 Desaturase Assays:

Human HepG2 cells were grown on 96-well plates in MEM media (Gibco cat#11095-072) supplemented with 10% heat-inactivated fetal bovine serum at 37° C. under 5% CO₂ in a humidified incubator. Test compound dissolved in the media was incubated with the sub-confluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of formula I, particularly the inhibitors of Examples 1 to 11, exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 to 11, is at least about ten or more, and preferably about one hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C. After phosphoric acid acidification of the extract, the amount of [¹⁴C]-stearic acid and [¹⁴C]-oleic acid was quantified on a HPLC system that was equipped with a C-18 reverse phase column and a Packard Flow Scintillation Analyzer.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred, particularly in combination with a pharmaceutically acceptable carrier. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Examples of other active ingredients that may be administered in combination with a compound of Formula I, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin and insulin analogs or derivatives, such as insulin lispro, insulin detemir, insulin glargine, insulin glulisine, and inhalable formulations of each thereof;

(4) leptin and leptin derivatives, agonists, and analogs, such as metreleptin;

(5) amylin; amylin analogs, such as davalintide; and amylin agonists, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics (See for example, WO 2008/011446, U.S. Pat. No. 5,545,618, U.S. Pat. No. 6,191,102, and US56583111); and GLP-1 receptor agonists, such as oxyntomodulin and its analogs and derivatives (See for example, WO 2003/022304, WO 2006/134340, WO 2007/100535), glucagon and its analogs and derivatives (See for example, WO 2008/101017), exenatide, liraglutide, taspoglutide, albiglutide, AVE0010, CJC-1134-PC, NN9535, LY2189265, LY2428757, and BIM-51077, including intranasal, transdermal, and once-weekly formulations thereof, such as exenatide QW;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-116, GPR-119, and GPR-40;

(22) SSTR3 antagonists, such as those disclosed in WO 2009/011836;

(23) neuromedin U receptor 1 (NMUR1) and/or neuromedin U receptor 2 (NMUR2) agonists, such as those disclosed in WO2007/109135 and WO2009/042053, including, but not limited to, neuromedin U (NMU) and neuromedin S (NMS) and their analogs and derivatives;

(24) GPR-105 (P2YR14) antagonists, such as those disclosed in WO 2009/000087;

(25) inhibitors of glucose uptake, such as sodium-glucose transporter (SGLT) inhibitors and its various isoforms, such as SGLT-1; SGLT-2, such as dapagliflozin and remogliflozin; and SGLT-3;

(26) inhibitors of acyl coenzyme A:diacylglycerol acyltransferase 1 and 2 (DGAT-1 and DGAT-2);

(27) inhibitors of fatty acid synthase;

(28) inhibitors of acyl coenzyme A:monoacylglycerol acyltransferase 1 and 2 (MGAT-1 and MGAT-2);

(29) agonists of the TGR5 receptor (also known as GPBAR1, BG37, GPCR19, GPR131, and M-BAR);

(30) bromocriptine mesylate and rapid-release formulations thereof;

(31) histamine H3 receptor agonists; and

(32) α2-adrenergic or β3-adrenergic receptor agonists.

Dipeptidyl peptidase-IV (DPP-4) inhibitors that can be used in combination with compounds of Formula I include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of Formula I include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl) tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of Formula I include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); histamine 1-13 receptor inverse agonists; 5-hydroxytryptamine-2c (5-HT2c) agonists, such as lorcaserin; and inhibitors of fatty acid synthase (FAS). For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of Formula I include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Agonists of the GPR-119 receptor that can be used in combination with the compounds of Formula I include, but are not limited to:
rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and
   pharmaceutically acceptable salts thereof.

Selective PPARγ modulators (SPPARγM's) that can be used in combination with the compounds of Formula I include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and
   pharmaceutically acceptable salts and esters thereof.

Inhibitors of 11β-hydroxysteroid dehydrogenase type 1 that can be used in combination with the compounds of Formula I include, but are not limited to:
3-[1-(4-chlorophenyl)-trans-3-fluorocyclobutyl]-4,5-dicyclopropyl-r-4H-1,2,4-triazole;
3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and
   pharmaceutically acceptable salts thereof.

Somatostatin subtype receptor 3 (SSTR3) antagonists that can be used in combination with the compounds of Formula I include, but are not limited to:

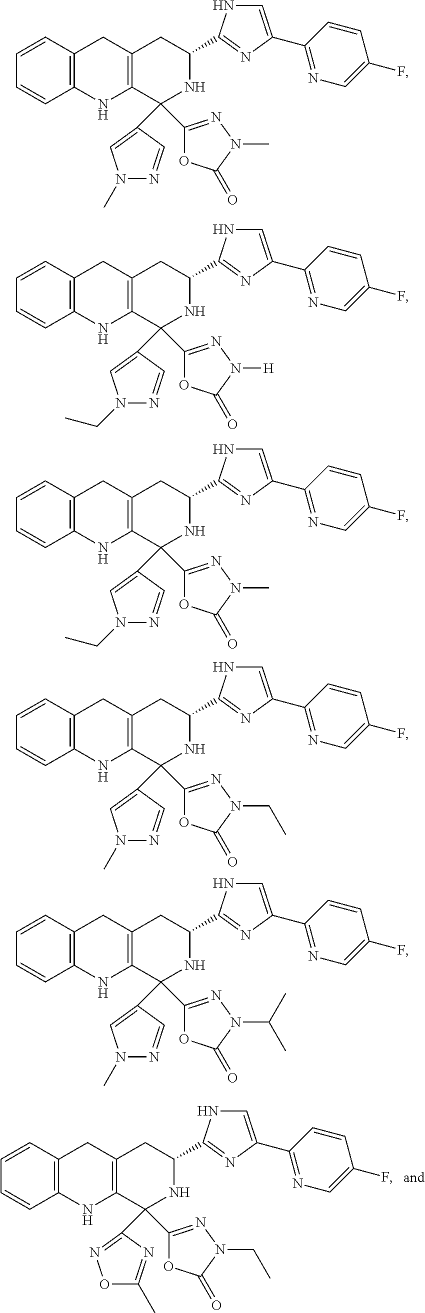

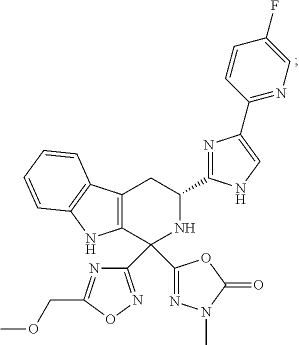

and pharmaceutically acceptable salts thereof.

AMP-activated Protein Kinase (AMPK) activators that can be used in combination with the compounds of Formula I include, but are not limited to:

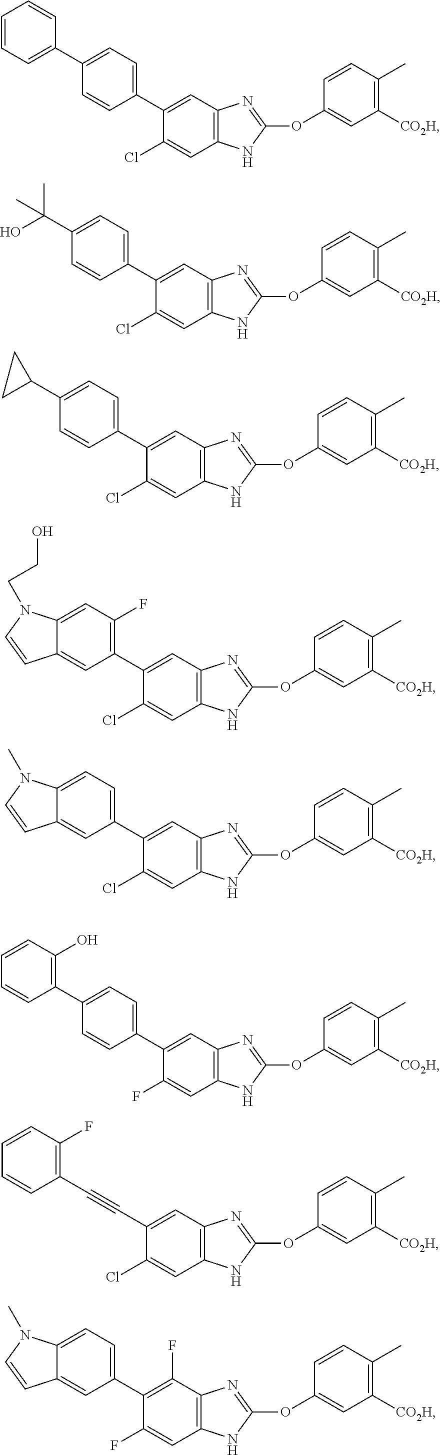

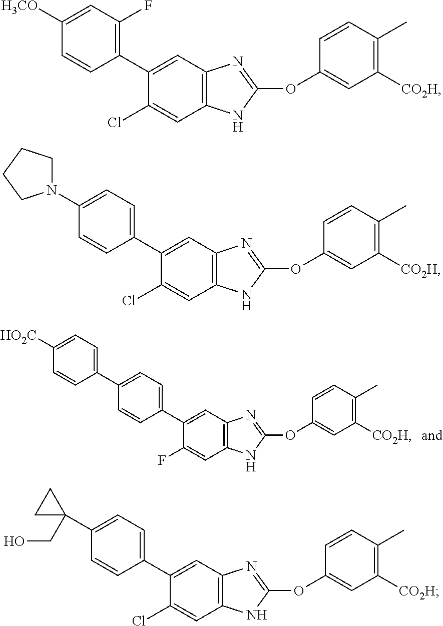

and pharmaceutically acceptable salts and esters thereof.

Inhibitors of acetyl-CoA carboxylase-1 and 2 (ACC-1 and ACC-2) that can be used in combination with the compounds of Formula I include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one;
5-{1′-[(1-cyclopropyl-4-methoxy-3-methyl-1H-indol-6-yl)carbonyl]-4-oxo-spiro[chroman-2,4′-piperidin]-6-yl}nicotinic acid;
4′-({6-(5-carbamoylpyridin-2-yl)-4-oxospiro[chroman-2,4′-piperidin]-1′-yl}carbonyl)-2′,6′-diethoxybiphenyl-4-carboxylic acid;
2′,6′-diethoxy-4′-{[6-(1-methyl-1H-pyrazol-4-yl)-4-oxospiro[chroman-2,4′-piperidin]-1′-yl]carbonyl}biphenyl-4-carboxylic acid;
2′,6′-diethoxy-3-fluoro-4′-{[6-(1-methyl-1H-pyrazol-4-yl)-4-oxospiro[chroman-2,4′-piperidin]-1′-yl]carbonyl}biphenyl-4-carboxylic acid;
5-[4-({6-(3-carbamoylphenyl)-4-oxospiro[chroman-2,4′-piperidin]-1′-yl}carbonyl)-2,6-diethoxyphenyl]nicotinic acid;
sodium 4′-({6-(5-carbamoylpyridin-2-yl)-4-oxospiro[chroman-2,4′-piperidin]-1′-yl}carbonyl)-2′,6′-diethoxybiphenyl-4-carboxylate;
methyl 4′-({6-(5-carbamoylpyridin-2-yl)-4-oxospiro[chroman-2,4′-piperidin]-1′-yl}carbonyl)-2′,6′-diethoxybiphenyl-4-carboxylate;
1′-[(4,8-dimethoxyquinolin-2-yl)carbonyl]-6-(1H-tetrazol-5-yl)spiro[chroman-2,4′-piperidin]-4-one;
(5-{1′-[(4,8-dimethoxyquinolin-2-yl)carbonyl]-4-oxospiro[chroman-2,4′-piperidin]-6-yl}-2H-tetrazol-2-yl)methyl pivalate;
5-{1′-[(8-cyclopropyl-4-methoxyquinolin-2-yl)carbonyl]-4-oxospiro[chroman-2,4′-piperidin]-6-yl}nicotinic acid;
1′-(8-methoxy-4-morpholin-4-yl-2-naphthoyl)-6-(1H-tetrazol-5-yl)spiro[chroman-2,4′-piperidin]-4-one; and
1′-[(4-ethoxy-8-ethylquinolin-2-yl)carbonyl]-6-(1H-tetrazol-5-yl)spiro[chroman-2,4′-piperidin]-4-one; and
   pharmaceutically acceptable salts and esters thereof.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequalae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention

The compounds of structural formula I can be prepared according to the procedures of the following Schemes and Examples, using appropriate materials and are further exemplified by the following specific example. The compound illustrated in the example is not, however, to be construed as forming the only genus that is considered as the invention. The Example further illustrates details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### LIST OF ABBREVIATIONS

ACN=acetonitrile
CuSO₄=copper sulfate
DBU=1,8-diazabicyclo[5.4.0]undec-7-ene
DCM=dichloromethane

### DMF=N,N-dimethylformamide

ESI=electrospray ionization
Et₃N=triethylamine
EtOAc=ethyl acetate
Et₂O=diethyl ether
h=hour(s)
HCl=hydrochloric acid
K₂CO₃=potassium carbonate
LC=liquid chromatography
MeOH=methyl alcohol
MgSO₄=magnesium sulfate
Min=minute(s)
MS=mass spectrum
MTBE=methyl tert-butyl ether
NaOH=sodium hydroxide
NaN₃=sodium azide
NMR=nuclear magnetic resonance spectroscopy
SiO₂=silicon dioxide
TFAA=trifluoroacetic anhydride
THF=tetrahydrofuran

### Method A:

An appropriately substituted bicyclic heterocyclic halide 1 (L=Cl, Br, or I) is converted to the acetylene intermediate 2 according to well-established literature procedures such as the Sonogashira reaction. The acetylene intermediate 2 is then reacted with various 1,3-dipoles, such as an azide and a nitrite oxide, to give a 1,3-cycloaddition adduct, which may require additional steps such as deprotection or other transformations to afford the final product of structural formula (I).

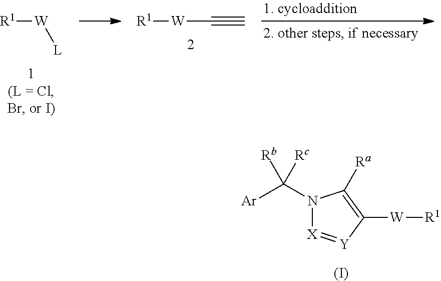

### Method B:

The precursor acetylene 3 (U═SnR₃, B(OR)₂) is reacted with various 1,3-dipoles, such as an azide and a nitrile oxide, to give the 1,3-cycloaddition adduct 4. Then, an appropriately substituted bicyclic heterocyclic halide 1 (L=Cl, Br, or I) is reacted with intermediate 4 according to well-established literature procedures, such as the Suzuki and Stille reaction. Additional steps such as deprotection or other transformations may be necessary to obtain the final product of structural formula (I).

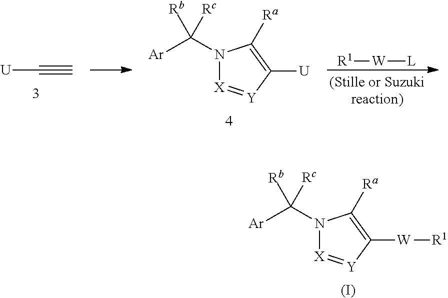

### Method C:

An appropriately substituted acetylenic ketone 5 can be reacted with various 1,3-dipoles, such as azide and a nitrile oxide, to give the 1,3-cycloaddition adduct 6. Ketone 6 can be converted to the corresponding bromo ketone 7 by reaction with bromine. Reaction of bromo ketone 7 with an appropriately substituted 2-aminopyridine 8 affords the desired imidazopyridine 9.

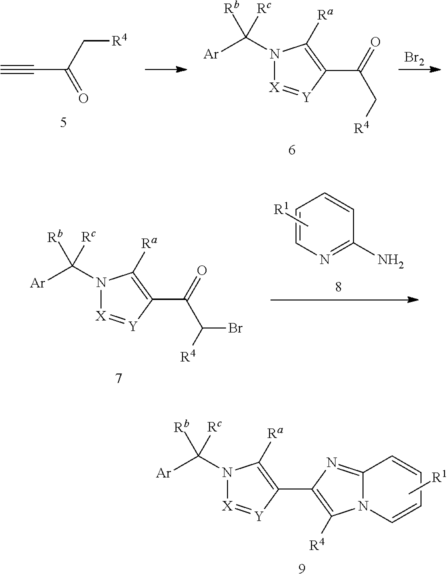

The following Examples are provided to illustrate the invention and are not to be construed as limiting the scope of the invention in any manner.

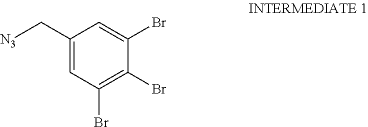

### 3,4,5-Tribromobenzyl azide

### Step 1

### 1,2,3-Tribromo-5-methylbenzene

To a mixture of CuBr₂ (0.44 g, 2.0 mmol) in MeCN (40 mL) at 50° C. was added a solution of Br₂ (1.9 mL, 37 mmol) in MeCN (10 mL) followed by the addition of p-toluidine (1.07 g, 10.0 mmol) in MeCN (5 mL). After stirring at 50° C. for 1 h, a solution of t-BuONO (1.43 mL, 37 mmol) in MeCN (20 mL) was added dropwise over 15 min. After stirring at 50° C. for 0.5 h, the reaction mixture was cooled to RT, and 20 mL of saturated aqueous Na₂SO₃ solution was added. The mixture was poured into 200 mL of 3M HCl and extracted with petroleum ether (2×200 mL). The combined organic layers were washed with 3M HCl (100 mL) and brine (100 mL), dried over Na₂SO₄ and concentrated. The crude product was dissolved in petroleum ether (50 mL) and purified by chromatography over silica gel (20 g) and eluted with petroleum ether (150 mL). The combined fractions were concentrated in vacuum to afford the product as a white solid.

¹H NMR (400 MHz, CDCl₃): δ 7.40 (s, 2H), 2.27 (s, 3H).

### Step 2

### 1,2,3-Tribromo-5-(bromomethyl)benzene

To a mixture of 1,2,3-tribromo-5-methylbenzene (5.00 g, 15.3 mmol) in CCl₄ (100 mL) was added NBS (0.55 g, 3.1 mmol) and (PhCOO)₂ (0.10 g, 0.41 mmol) and the mixture was heated at 80-90° C. for 3 h. Two additional portions of NBS (0.55 g, 3 mmol) and (PhCOO)₂ (0.1 g, 0.4 mmol) were added at 4 and 5 h. The resulting mixture was heated overnight. After cooling to RT, the mixture was filtered over silica gel (10 g) and concentrated. The crude product was suspended in petroleum ether (50 mL) and stirred for 5 min. The resulting white solid was collected by vacuum filtration to afford the title product.

¹H NMR (300 MHz, CDCl₃): δ 7.62 (s, 2H), 4.34 (s, 2H).

### Step 3

### 3,4,5-Tribromobenzyl azide

To a solution of 1,2,3-tribromo-5-(bromomethyl)benzene (0.50 g, 1.24 mmol) in DMF (10 mL) was added sodium azide (0.12 g, 1.9 mmol). After stirring for 1 h at RT, the mixture was poured over water (60 mL) and was extracted with petroleum ether (2×20 mL). The combined organic layers were washed with brine (2×20 mL), dried over Na₂SO₄ and concentrated to afford the title product.

¹H NMR (400 MHz, CDCl₃): δ 7.57 (s, 2H), 4.32 (s, 2H).

### Intermediate 2

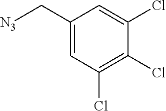

### 3,4,5-Trichlorobenzyl azide

### Step 1

### (4-Amino-3,5-dichlorophenyl)methanol

To a solution of 4-amino-3,5-dichlorobenzoic acid (10 g, 48.5 mmol) in THF (243 mL) was added lithium aluminum hydride (10 g, 48.5 mmol) in small portions over 15 min at 0° C. The mixture was warmed to RT and stirred for 3 h, then heated at 60° C. for 7 h. The mixture was cooled to 0° C. and carefully quenched with 15% NaOH (4.1 mL). The solid was filtered and washed with EtOAc (30 mL). The mother liquor was evaporated and the crude product was recrystallized from Et₂O/Hexanes to afford the title product as a solid.

¹H NMR (500 MHz, acetone-d₆): δ 7.21 (s, 2H), 5.04 (s, 2H), 4.48 (d, 2H), 4.18 (t, 1 H). MS (+ESI) m/z 192, 194 (MH⁺).

### Step 2

### (3,4,5-Trichlorophenyl)methanol

To a mixture of (4-amino-3,5-dichlorophenyl)methanol (6.6 g, 34.4 mmol) in acetonitrile (86 mL) was added copper(II) chloride (5.54 g, 41.2 mmol). After 5 min, tert-butyl nitrite (6.80 mL, 51.6 mmol) was added and the mixture stirred at RT for 1.5 h. The solvent was evaporated under reduced pressure and the residue was diluted with 2N HCl (50 ml) and extracted with Et₂O (3×25 mL). The combined organic fractions were washed with water (25 mL) then dried over MgSO₄. The solvent was evaporated under reduced pressure and the product was triturated with DCM/hexanes, filtered and washed with hexanes to afford the title compound as a solid.

¹H NMR (500 MHz, acetone-d₆): δ 7.54 (s, 2H), 4.65 (s, 2H).

### Step 3

### 3,4,5-Trichlorobenzyl azide

To a solution of (3,4,5-trichlorophenyl)methanol (2.6 g, 12.29 mmol) and diphenylphosphoryl azide (3.20 ml, 14.75 mmol) in toluene (24.6 mL) was added DBU (2.04 mL, 13.52 mmol). The reaction mixture was stirred at RT for 3.5 h. The mixture was diluted with 1N HCl (25 mL). The organic layer was separated and dried over MgSO₄. Purification by Combiflash™ chromatography (SiO₂-40 g, elution with 100% hexanes over 10 min) afforded the title compound as an oil.

¹H NMR (500 MHz, acetone-d₆): δ 7.62 (s, 2H), 4.55 (s, 2H).

### Intermediate 3

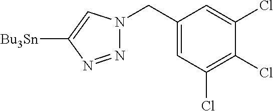

### 4-(Tributylstannyl)-1-(3,4,5-trichlorobenzyl)-1H-1,2,3-triazole

To a solution of 3,4,5-trichlorobenzyl azide (100 mg, 0.423 mmol) (Intermediate 2) in benzene (1 mL) was added ethynyl tri-n-butyltin (247 μL, 0.486 mmol). The reaction mixture was stirred at 80° C. for 10 h. The solvent was evaporated under reduced pressure and the residue was purified by Combiflash chromatography, (SiO₂-12 g, elution with 0-10% EtOAc/hexanes over 30 min) afforded the title compound as the major regioisomer.

¹H NMR (500 MHz, CDCl₃): δ 7.46 (s, 1H), 7.26 (s, 2H), 5.53 (s, 2H), 1.56 (m, 6H), 1.34 (h, 6H), 1.14 (t, 6H), 0.89 (t, 9H). MS (+ESI) m/z 552 (MH⁺).

### Example 1

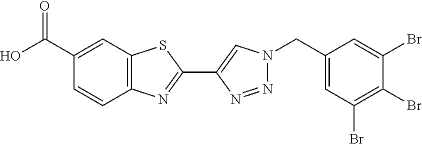

### 2-[1-(3,4,5-Tribromobenzyl)-1H-1,2,3-triazol-4-yl]-1,3-benzothiazole-6-carboxylic acid

### Step 1

### Ethyl 2-bromo-1,3-benzothiazole-6-carboxylate

A suspension of CuBr₂ (1.20 g, 5.40 mmol) in anhydrous acetonitrile (15 mL) was purged with nitrogen. The mixture was cooled in an ice-bath and treated with t-BuONO (696 mg, 6.75 mmol). After further stirring for 10 min at about 0 to 5° C., the mixture was reacted with 2-amino-1,3-benzothiazole-6-carboxylate (1.0 g, 4.5 mmol). The cooling bath was removed and the reaction mixture was stirred at room temperature for 2 h. The mixture was then diluted with water (30 mL) and extracted with ether (100 mL×2). The combined organic phase was filtered to removed copper salts, then washed with water (20 mL) and brine (20 mL), dried over Na₂SO₄ and concentrated to afford the title compound.

¹H NMR (CDCl₃, 400 MHz): δ 8.54 (d, 1H), 8.16 (dd, 1H), 8.02 (d, 1H), 4.42 (q, 2H), 1.42 (t, 3H).

### Step 2

### Ethyl 2-[(trimethylsilyl)ethynyl]-1,3-benzothiazole-6-carboxylate

To a stirred solution of ethyl 2-bromo-1,3-benzothiazole-6-carboxylate (880 mg, 3.07 mmol) and ethynyltrimethylsilane (453 mg, 4.61 mmol) in toluene (15 mL) and TEA (5 mL) was added CuI (117 mg, 0.61 mmol), Pd(PPh₃)₂Cl₂ (216 mg, 0.31 mmol) and PPh₃ (81 mg, 0.31 mmol) at room temperature under nitrogen atmosphere. The reaction mixture was stirred at 60° C. for 4 h. After cooling, the mixture was filtered and the solid was washed with EtOAc (200 mL). The combined organic phase was concentrated. The crude residue was purified by silica gel column chromatography (PE/EA=50:1) to afford the title compound.

¹H NMR (CDCl₃, 400 MHz): δ 8.58 (d, 1H), 8.11 (dd, 1H), 8.01 (d, 1H), 4.36 (q, 2H), 1.42 (t, 3H), 0.32 (s, 9H).

### Step 3

### Ethyl 2-ethynyl-1,3-benzothiazole-6-carboxylate

To a stirred solution of ethyl 2-[(trimethylsilyl)ethynyl]-1,3-benzothiazole-6-carboxylate (50 mg, 0.16 mmol) and 18-crown-6 (44 mg, 0.16 mmol) in 4 mL of DCM was added KF (19 mg, 0.33 mmol) under ice-bath. The ice-bath was then removed and the reaction mixture was stirred at room temperature for 30 min. More DCM (30 mL) was added and the resulting solution was washed with water (10 mL). The organic layer was dried over Na₂SO₄ and concentrated in vacuum to afford the title compound.

¹H NMR (CDCl₃, 400 MHz): δ 8.60 (d, 1H), 8.20 (dd, 1H), 8.10 (d, 1H), 4.43 (q, 2H), 3.68 (s, 1H), 1.43 (t, 3H).

### Step 4

### Ethyl 2-[1-(3,4,5-tribromobenzyl)-1H-1,2,3-triazol-4-yl]-1,3-benzothiazole-6-carboxylate

A solution of ethyl 2-ethynyl-1,3-benzothiazole-6-carboxylate (300 mg, 1.29 mmol) and 3,4,5-tribromobenzyl azide (959 mg, 2.59 mmol) in 20 mL of toluene was refluxed overnight. After removing the solvent, the residue was purified by silica gel chromatography (DCM/EA=30:1) to afford the title compound and the regioisomer ethyl 2-[1-(3,4,5-tribromobenzyl)-1H-1,2,3-triazol-5-yl]-1,3-benzothiazole-6-carboxylate.

¹H NMR (CDCl₃, 400 MHz): δ 8.66 (s, 1H), 8.21 (s, 1H), 8.16 (dd, 1H), 8.00 (d, 1H), 7.55 (s, 2H), 5.52 (s, 2H), 4.41 (q, 2H), 1.42 (t, 3H).

### Step 5

### 2-[1-(3,4,5-Tribromobenzyl)-1H-1,2,3-triazol-4-yl]-1,3-benzothiazole-6-carboxylic acid

A mixture of ethyl 2-[1-(3,4,5-tribromobenzyl)-1H-1,2,3-triazol-4-yl]-1,3-benzothiazole-6-carboxylate (160 mg, 0.26 mmol) and 1 M NaOH (0.55 mL, 0.55 mmol) in THF (8 mL) and MeOH (2 mL) was stirred at room temperature overnight. Volatile materials were removed under vacuum. The residue was diluted with water (10 mL), acidified to pH=1 with 1 M HCl and extracted with EtOAc (50 mL×2). The combined organic layers were washed with brine (20 mL), dried over Na₂SO₄ and concentrated to afford the title compound.

¹H NMR (DMSO-d₆, 400 MHz): δ 9.10 (s, 1H), 8.75 (s, 1H), 8.04 (s, 2H), 7.05 (s, 2H), 5.68 (s, 21-1). MS: m/z 573 and 575 (MH⁺).

### Example 2

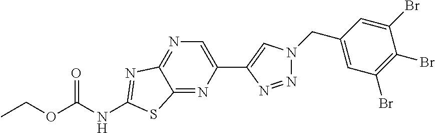

### Ethyl{6-[1-(3,4,5-tribromobenzyl)-1H-1,2,3-triazol-4-yl][1,3]thiazolo[4,5-b]pyrazin-2-yl}carbamate

### Step 1

### Ethyl{6-[(trimethylsilyl)ethynyl][1,3]thiazolo[4,5-b]pyrazin-2-yl}carbamate

To a degassed solution of ethyl (6-bromo[1,3]thiazolo[4,5-b]pyrazin-2-yl)carbamate (175 mg, 0.577 mmol) [prepared as described by Koren, B.; Stanovinik, B.; Tisler, M., in Heterocycles 1987, 3, 689] and Et₃N (0.6 mL) in DCM (1.3 mL) was added copper(I) iodide (8.79 mg, 0.046 mmol), Pd(Ph₃P)₄ (8.00 mg, 0.029 mmol) and trimethylsilylacetylene (74 μL, 0.750 mmol). The reaction mixture was warmed to 50° C. and stirred for 3 h. After cooling to RT, the mixture was filtered through celite and the filtrate was evaporated under reduced pressure. Purification by Combiflash™ chromatography, (SiO₂-12 g, elution with 0-65% EtOAc/hexanes over 40 min) afforded the title compound as a solid.

¹H NMR (500 MHz, acetone-d₆): δ 8.60 (s, 1H), 4.36 (q, 2H), 1.34 (t, 3H), 0.28 (s, 9H). MS (+ESI) m/z 322 (MH⁺).

### Step 2

### Ethyl (6-ethynyl[1,3]thiazolo[4,5-b]pyrazin-2-yl)carbamate

To a solution of ethyl{6-[(trimethylsilyl)ethynyl][1,3]thiazolo[4,5-b]pyrazin-2-yl}carbamate (140 mg, 0.438 mmol) in THF (6 mL) was added TBAF (438 μL, 0.438 mmol). The reaction mixture was stirred at RT for 0.5 h. The solvent was evaporated under reduced pressure. Purification by Combiflash™ chromatography (SiO₂-12 g, elution with 0-20% EtOAc/hexanes over 40 min) afforded the title compound as a solid.

¹H NMR (500 MHz, acetone-d₆): δ 8.63 (s, 1H), 4.35 (q, 2H), 4.03 (s, 1H), 1.34 (t, 3H). MS (+ESI) m/z 250 (MH⁺).

### Step 3

### Ethyl {6-[1-(3,4,5-tribromobenzyl)-1H-1,2,3-triazol-4-yl][1,3]thiazolo[4,5-b]pyrazin-2-yl}carbamate

A mixture of ethyl{6-[(trimethylsilyl)ethynyl][1,3]thiazolo[4,5-b]pyrazin-2-yl}carbamate (113 mg, 0.454 mmol), 3,4,5 trichlorobenzyl azide (140 mg, 0.379 mmol), L-ascorbic acid sodium salt (15.0 mg, 0.076 mmol) and copper(II) sulfate pentahydrate (9.5 mg, 0.038 mmol) in THF (1.3 mL) and water (0.6 mL) was heated at 60° C. for 1 h. The solvents were evaporated under reduced pressure. The residue was triturated with DCM/hexanes/water (1/10/1). The mixture was then filtered, washed with water and dried under vacuum. The residue was purified by trituration with DCM/Hexanes/MeOH to afford the title compound as a solid.

¹H NMR (500 MHz, acetone-d₆): δ 9.25 (d, 1H), 8.76 (s, 1H), 7.88 (s, 2H), 5.82 (s, 2H), 4.38 (q, 2H), 1.38 (t, 3H). MS (+ESI) m/z 618, 620 (MH⁺).

### Example 3

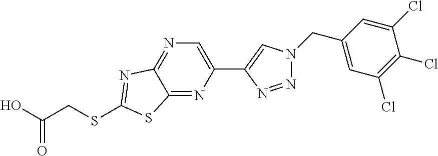

### ({6-[1-(3,4,5-Trichlorobenzyl)-1H-1,2,3-triazol-4-yl][1,3]thiazolo[4,5-b]pyrazin-2-yl}thio)acetic acid

### Step 1

### 2,6-Dibromo[1,3]thiazolo[4,5-b]pyrazine

To a solution of 6-bromo[1,3]thiazolo[4,5-b]pyrazin-2-amine (300 mg, 1.298 mmol) [prepared as described by Koren, B.; Stanovinik, B.; Tisler, M, in Heterocycles 1987, 3, 689] in acetonitrile (13.0 mL) was added copper(II) bromide (435 mg, 1.947 mmol). After 5 min, tert-butyl nitrite (0.308 mL, 2.59 mmol) was added. The reaction was stirred at RT for 18 h. The solvent was evaporated under reduced pressure and the residue was diluted with water (20 ml) and EtOAc (20 mL). The mixture was filtered through celite and the aqueous layer was extracted with EtOAc (3×75 mL). The combined organic fractions were washed with water (2×100 mL), dried (MgSO₄), filtered and the solvent was evaporated under reduced pressure to afford the title compound as a solid.

¹H NMR (500 MHz, acetone-d₆): δ 8.87 (s, 1H). MS (+ESI) m/z 292, 295 (MH⁺).

### Step 2

### Ethyl [(6-bromo[1,3]thiazolo[4,5-b]pyrazin-2-yl)thio]acetate and ethyl [(2-bromo[1,3]thiazolo[4,5-b]pyrazin-6-yl)thio]acetate

To a solution of 2,6-dibromo[1,3]thiazolo[4,5-b]pyrazine (50 mg, 0.170 mmol) in EtOH (0.848 mL) was added Et₃N (47.3 μL, 0.339 mmol) and ethyl 2-mercaptoacetate (20.37 μL, 0.186 mmol). The reaction mixture was stirred at RT for 30 min. The solvent was evaporated under reduced pressure and the residue was diluted with water (10 mL) and EtOAc (5 mL). The aqueous layer was extracted with EtOAc (3×15 mL) and the combined organic fractions were dried (MgSO₄), filtered and evaporated under reduced pressure to afford the title compounds as solids. The mixture of regioisomers was used directly in the next step without purification.

¹H NMR (500 MHz, acetone-d₆): δ 8.73 (s, 1H), 4.38 (s, 2H), 4.21 (q, 2H), 1.25 (t, 3H). MS (+ESI) m/z 333 (MH⁺).

### Step 3

### Ethyl({6-[1-(3,4,5-trichlorobenzyl)-1H-1,2,3-triazol-4-yl][1,3]thiazolo[4,5-b]pyrazin-2-yl}thio)acetate and ethyl ({2-[1-(3,4,5-trichlorobenzyl)-1H-1,2,3-triazol-4-yl][1,3]thiazolo[4,5-b]pyrazin-6-yl}thio)acetate

To a solution of 4-(tributylstannyl)-1-(3,4,5-trichlorobenzyl)-1H-1,2,3-triazole (150 mg, 0.272 mmol) (Intermediate 3) and a mixture of ethyl [(6-bromo[1,3]thiazolo[4,5-b]pyrazin-2-yl)thio]acetate and ethyl [(2-bromo[1,3]thiazolo[4,5-b]pyrazin-6-yl)thio]lacetate (100 mg, 0.299 mmol) in degassed dioxane (2.72 mL) was added bis(triphenylphosphine)palladium(II) chloride (19.09 mg, 0.027 mmol). The reaction mixture was heated at 110° C. for 5 h. The solvent was evaporated under reduced pressure. The residue was adsorbed on silica gel and purified by Combiflash™ chromatography (SiO₂-10 g, elution with 20-70% EtOAc/hexanes over 40 min) to afford a mixture of regioisomers. The regioisomers were separated using a Chiralpak AD™ column (4.6×250 mm), eluting with 40% IPrOH/hexanes with 0.25% TEA to afford the title compounds:

Ethyl ({6-[1-(3,4,5-trichlorobenzyl)-1H-1,2,3-triazol-4-yl][1,3]thiazolo[4,5-b]pyrazin-2-yl}thio)acetate was the major and more polar regioisomer.

¹H NMR (500 MHz, acetone-d₆): δ 9.32 (s, 1H), 8.79 (s, 1H), 7.73 (s, 2H), 5.86 (s, 2H), 4.43 (s, 2H), 4.25 (q, 2H), 1.30 (t, 3H). MS (+ESI) m/z 515, 517 (MH⁺).

Ethyl ({2-[1-(3,4,5-trichlorobenzyl)-1H-1,2,3-triazol-4-yl][1,3]thiazolo[4,5-b]pyrazin-6-yl}thio)acetate was the minor and less polar regioisomer.

¹H NMR (500 MHz, acetone-d₆): δ 8.98 (s, 1H), 8.71 (s, 1H), 7.78 (s, 2H), 5.92 (s, 2H), 4.22 (q, 2H), 4.18 (s, 2H), 1.27 (t, 3H). MS (+ESI) m/z 515, 517 (MH⁺).

### Step 4

### ({6-[1-(3,4,5-Trichlorobenzyl)-1H-1,2,3-triazol-4-yl][1,3]thiazolo[4,5-b]pyrazin-2-yl}thio)acetic acid

To a solution of ethyl ({6-[1-(3,4,5-trichlorobenzyl)-1H-1,2,3-triazol-4-yl][1,3]thiazolo[4,5-b]pyrazin-2-yl}thio)acetate (13 mg, 0.025 mmol) in THF (126 μL) was added 1N NaOH (76 μL, 0.076 mmol). The reaction mixture was stirred at RT for 1.5 h. The volatiles were evaporated under reduced pressure. Water was added (10 mL) and the aqueous layer was acidified with 1N HCl (pH about 1) and extracted with EtOAc (3×5 mL). The combined organic fractions were dried over MgSO₄, filtered and evaporated under reduced pressure. The residue was triturated with DCM/Hexanes (1/10) to afford the title compound as a solid.

¹H NMR (500 MHz, acetone-d₆): δ 9.30 (s, 1H), 8.77 (s, 1H), 7.73 (s, 2H), 5.85 (s, 2H), 4.39 (s, 2H). MS (+ESI) m/z 487, 489 (MH⁺).

### Example 4

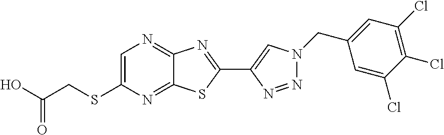

### ({2-[1-(3,4,5-Trichlorobenzyl)-1H-1,2,3-triazol-4-yl][1,3]thiazolo[4,5-b]pyrazin-6-yl}thio)acetic acid

The title compound was prepared in a similar manner as that described for Example 3 (step 4) from ethyl ({2-[1-(3,4,5-trichlorobenzyl)-1H-1,2,3-triazol-4-yl][1,3]thiazolo[4,5-b]pyrazin-6-yl}thio)acetate (Example 3, step 3, minor regioisomer).

¹H NMR (500 MHz, acetone-d₆): δ 8.96 (s, 1H), 8.69 (s, 1H), 7.77 (s, 2H), 5.91 (s, 2H), 4.19 (s, 2H). MS (+ESI) m/z 487, 489 (MH⁺).

### Example 5

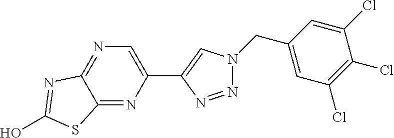

### 6-[1-(3,4,5-Trichlorobenzyl)-1H-1,2,3-triazol-4-yl][1,3]thiazolo[4,5-b]pyrazin-2-ol

### Step 1

### 2-[(6-Bromo[1,3]thiazolo[4,5-b]pyrazin-2-yl)thio]ethyl acetate and 2-[(2-bromo[1,3]thiazolo[4,5-b]pyrazin-6-yl)thio]ethyl acetate

The title compounds were prepared in a similar manner as that described for Example 3 (step 2) from 2,6-dibromo[1,3]thiazolo[4,5-b]pyrazine (Example 3, step 1) and 2-mercaptoethyl acetate. The mixture of regioisomers was used directly in the next step without purification.

¹H NMR (500 MHz, acetone-d₆): δ 8.76 (s, 1H), 4.49 (t, 2H), 3.81 (t, 2H), 2.04 (s, 3 H). MS (+ESI) m/z 356, 358 (M+Na⁺).

### Step 2

### 2-({6-[1-(3,4,5-Trichlorobenzyl)-1H-1,2,3-triazol-4-yl][1,3]thiazolo[4,5-b]pyrazin-2-yl}thio)ethyl acetate and 2-({2-[1-(3,4,5-trichlorobenzyl)-1H-1,2,3-triazol-4-yl][1,3]thiazolo[4,5-b]pyrazin-6-yl}thio)ethyl acetate

The title compounds were prepared in a similar manner as that described for Example 3 (step 3) from the mixture of 2-[(6-bromo[1,3]thiazolo[4,5-b]pyrazin-2-yl)thio]ethyl acetate and 2-[(2-bromo[1,3]thiazolo[4,5-b]pyrazin-6-yl)thio]ethyl acetate with 4-(tributylstannyl)-1-(3,4,5-trichlorobenzyl)-1H-1,2,3-triazole (Intermediate 3). The regioisomers were separated using a Chiralpak AD™ column (4.6×250 mm) eluting with 40% iPrOH/hexanes with 0.25% TEA to afford the title compounds:

2-({6-[1-(3,4,5-Trichlorobenzyl)-1H-1,2,3-triazol-4-yl][1,3]thiazolo[4,5-b]pyrazin-2-yl}thio)ethyl acetate was obtained as the major and more polar regioisomer.

¹H NMR (500 MHz, acetone-d₆): δ 9.32 (s, 1H), 8.77 (s, 1H), 7.72 (s, 2H), 5.85 (s, 2H), 4.50 (t, 2H), 3.81 (t, 2H), 2.03 (s, 3H). MS (+ESI) m/z 515, 517 (MH⁺).

2-({2-[1-(3,4,5-Trichlorobenzyl)-1H-1,2,3-triazol-4-yl][1,3]thiazolo[4,5-b]pyrazin-6-yl}thio)ethyl acetate was obtained as the minor and less polar regioisomer.

¹H NMR (500 MHz, acetone-d₆): δ 8.94 (s, 1H), 8.61 (s, 1H), 7.74 (s, 2H), 5.88 (s, 2H), 4.35 (t, 2H), 3.58 (t, 2H), 1.98 (s, 3H). MS (+ESI) m/z 515, 517 (MH⁺).

### Step 3

### 6-[1-(3,4,5-Trichlorobenzyl)-1H-1,2,3-triazol-4-yl][1,3]thiazolo[4,5-b]pyrazin-2-ol

The title compound was prepared in a similar manner as that described for Example 3 (step 4) from 2-({6-[1-(3,4,5-trichlorobenzyl)-1H-1,2,3-triazol-4-yl][1,3]thiazolo[4,5-b]pyrazin-2-yl}thio)ethyl acetate and NaOH to afford the final product.

1H NMR (500 MHz, acetone-d₆): δ 8.88 (s, 1H), 8.64 (s, 1H), 7.70 (s, 2H), 5.82 (s, 3H). MS (+ESI) m/z 413, 415 (MH⁺).

### Example 6

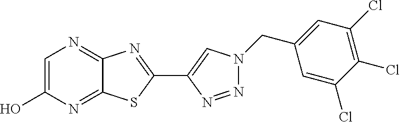

### 2-[1-(3,4,5-Trichlorobenzyl)-1H-1,2,3-triazol-4-yl][1,3]thiazolo[4,5-b]pyrazin-6-ol

The title compound was prepared in a similar manner as that described for Example 3 (step 4) from 2-({2-[1-(3,4,5-trichlorobenzyl)-1H-1,2,3-triazol-4-yl][1,3]thiazolo[4,5-b]pyrazin-6-yl}thio)ethyl acetate (Example 5, step 2, minor regioisomer) and NaOH to afford the final product.

¹H NMR (500 MHz, acetone-d₆): δ 8.87 (s, 1H), 8.26 (s, 1H), 7.76 (s, 2H), 5.89 (s, 2H). MS (+ESI) m/z 437, 439 (MH⁺).

### Example 7

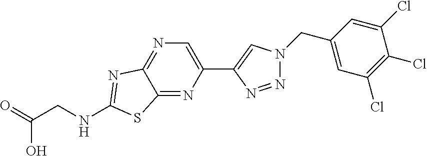

### ({6-[1-(3,4,5-Trichlorobenzyl)-1H-1,2,3-triazol-4-yl][1,3]thiazolo[4,5-b]pyrazin-2-yl}amino)acetic acid

### Step 1

### Methyl [(6-bromo[1,3]thiazolo[4,5-b]pyrazin-2-yl)amino]acetate

To a solution of 2,6-dibromo[1,3]thiazolo[4,5-b]pyrazine (50 mg, 0.170 mmol) (Example 3, step 1) in EtOH (848 μL) was added Et₃N (70.9 μL, 0.509 mmol) followed by methyl aminoacetate hydrochloride (23.41 mg, 0.186 mmol). The reaction was stirred at RT for 6 h. The solvent was evaporated under reduced pressure and the residue was diluted with water (10 mL). The aqueous layer was extracted with EtOAc (3×5 mL) and the combined organic fractions were dried (MgSO₄), filtered and evaporated under reduced pressure to afford the title compound as a solid.

¹H NMR (500 MHz, acetone-d₆): δ 8.39 (s, 1H), 4.43 (d, 2H), 3.73 (s, 3H). MS (+ESI) m/z 303, 305 (MH⁺).

### Step 2

### Methyl ({6-[1-(3,4,5-trichlorobenzyl)-1H-1,2,3-triazol-4-yl][1,3]thiazolo[4,5-b]pyrazin-2-yl}amino)acetate

The title compound was prepared in a similar manner as that described for Example 3 (step 3) from methyl [(6-bromo[1,3]thiazolo[4,5-b]pyrazin-2-yl)amino]acetate and 4-(tributylstannyl)-1-(3,4,5-trichlorobenzyl)-1H-1,2,3-triazole (Intermediate 3).

¹H NMR (500 MHz, acetone-d₆): δ 9.03 (s, 1H), 8.60 (s, 1H), 7.70 (s, 2H), 5.81 (s, 2H), 4.46 (s, 2H), 3.77 (s, 3H). MS (+ESI) m/z 485, 487 (MH⁺).

### Step 3

### 2({6-[1-(3,4,5-Trichlorobenzyl)-1H-1,2,3-triazol-4-yl][1,3]thiazolo[4,5-b]pyrazin-2-yl}amino)acetic acid

The title compound was prepared in a similar manner as that described for Example 3 (step 4) from methyl ({6-[1-(3,4,5-trichlorobenzyl)-1H-1,2,3-triazol-4-yl][1,3]thiazolo[4,5-b]pyrazin-2-yl}amino)acetate and NaOH.

¹H NMR (500 MHz, acetone-d₆): δ 9.01 (d, 1H), 8.59 (d, 1H), 7.70 (s, 2H), 5.81 (s, 2H), 4.41 (s, 2H). MS (+ESI) m/z 470, 472 (MH⁺).

### Example 8

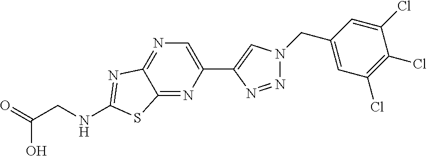

### ({5-[1-(3,4,5-Trichlorobenzyl)-1H-1,2,3-triazol-4-yl][1,3]thiazolo[5,4-d]pyrimidin-2-yl}amino)acetic acid

### Step 1

### Methyl [(5-chloro[1,3]thiazolo[5,4-d]pyrimidin-2-yl)amino]acetate

2,4-Dichloropyrimidin-5-amine (300 mg, 1.829 mmol) and ethyl isothiocyanatoacetate (1.27 mL, 10.24 mmol) were heated in a flask at 100° C. for 10 min. MeOH (4.0 mL) was then added and the temperature was adjusted to 75° C. After 18 h, the solvent was evaporated under reduced pressure. Diethyl ether was added (5 mL) to the residue and the solid was filtered, then washed with diethyl ether. The mother liquor was evaporated and trituration of the residue with diethyl ether/hexanes (1/10) afforded the title compound as a solid.

¹H NMR (500 MHz, acetone-d₆): δ 8.57 (s, 1H), 8.22 (bs, 1H), 4.41 (s, 2H), 4.36 (s, 2H). MS (+ESI) m/z 259 (MH⁺).

### Step 2

### Methyl ({5-[1-(3,4,5-trichlorobenzyl)-1H-1,2,3-triazol-4-yl][1,3]thiazolo[5,4-d]pyrimidin-2-yl}amino)acetate

The title compound was prepared in a similar manner as that described for Example 3 (step 3) from methyl [(5-chloro[1,3]thiazolo[5,4-d]pyrimidin-2-yl)amino]acetate and 4-(tributylstannyl)-1-(3,4,5-trichlorobenzyl)-1H-1,2,3-triazole (Intermediate 3).

¹H NMR (500 MHz, acetone-d₆): δ 8.71 (s, 1H), 8.68 (s, 1H), 7.71 (s, 2H), 5.82 (s, 2H), 4.43 (s, 2H), 3.77 (s, 3H). MS (+ESI) m/z 484, 486 (MH⁺).

### Step 3

### ({5-[1-(3,4,5-Trichlorobenzyl)-1H-1,2,3-triazol-4-yl][1,3]thiazolo[5,4-d]pyrimidin-2-yl}amino)acetic acid

The title compound was prepared in the same manner as described in Example 4, step 5 from methyl ({5-[1-(3,4,5-trichlorobenzyl)-1H-1,2,3-triazol-4-yl][1,3]thiazolo[5,4-d]pyrimidin-2-yl}amino)acetate and NaOH.

¹H NMR (500 MHz, acetone-d₆): δ 8.70 (s, 1H), 8.68 (s, 1H), 7.71 (s, 2H), 5.81 (s, 2H), 4.35 (s, 2H). MS (+ESI) m/z 470, 472 (MH⁺).

### Example 9

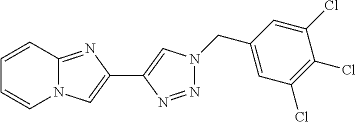

### 2-[1-(3,4,5-Trichlorobenzyl)-1H-1,2,3-triazol-4-yl]imidazo[1,2-a]pyridine

### Step 1

### 1-[1-(3,4,5-Trichlorobenzyl)-1H-1,2,3-triazol-4-yl]ethanone

A mixture of 3,4,5-trichlorobenzyl azide (Intermediate 2) (1 g, 4.23 mmol), 3-butyn-2-one (0.364 mL, 4.65 mmol), L-ascorbic acid sodium salt (0.168 g, 0.846 mmol) and copper(II) sulfate pentahydrate (0.106 g, 0.423 mmol) in THF (7 mL) and water (3.5 mL) was heated at 60° C. for 12 h. The THF was evaporated and the mixture was slurried with 1N HCl (5 mL) and hexanes (5 mL). The mixture was filtered and washed with water followed by hexanes. The solid was dried under high vacuum to afford the title product.

¹H NMR (500 MHz, acetone-d₆): δ 8.64 (s, 1H), 7.68 (s, 2H), 5.81 (s, 2H), 2.58 (s, 3H). MS: m/z 304, 306 (MH⁺).

### Step 2

### 2-Bromo-1-[1-(3,4,5-trichlorobenzyl)-1H-1,2,3-triazol-4-yl]ethanone

To a solution of 1-[1-(3,4,5-trichlorobenzyl)-1H-1,2,3-triazol-4-yl]ethanone (350 mg, 1.149 mmol) in THF (2.9 mL) and acetic acid (2.9 mL) was added 30% HBr in AcOH (0.21 mL, 1.149 mmol) followed by bromine (0.07 mL, 1.264 mmol). The mixture was stirred at RT for 6 h. The acetic acid was evaporated, the residue was diluted with water (4 mL) and extracted with EtOAc (3×3 mL). The combined organic fractions were dried over Na₂SO₄ and the solvent was evaporated. Purification by Combiflash™ chromatography (SiO₂-40 g, gradient elution of 10-30% EtOAc/hexanes over 30 min) afforded the title product as a solid. MS: m/z 384, 386 (MH⁺).

### Step 3

### 2-[1-(3,4,5-Trichlorobenzyl)-1H-1,2,3-triazol-4-yl]imidazo[1,2-a]pyridine

A mixture of 2-aminopyridine (13.5 mg, 0.143 mmol) and 2-bromo-1-[1-(3,4,5-trichlorobenzyl)-1H-1,2,3-triazol-4-yl]ethanone (50 mg, 0.130 mmol) in EtOH (1.304 mL) was heated at 100° C. for 3 h. The mixture was diluted with saturated NaHCO₃ (2 mL) and heated again at 100° C. for 0.5 h. The mixture was cooled to RT and diluted with water (2 mL). The solid was filtered and washed with water followed by Et₂O. The solid was dried under high vacuum to afford the title product.

¹H NMR (500 MHz, acetone-d₆): δ 8.55 (d, 1H), 8.44 (s, 1H), 8.28 (s, 1H), 7.68 (s, 2H), 7.51 (d, 1H), 7.29-7.25 (m, 1H), 6.91 (t, 1H), 5.79 (s, 2H). MS: m/z 378, 380 (MH⁺).

### Example 10

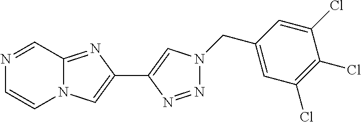

### 2-[1-(3,4,5-Trichlorobenzyl)-1H-1,2,3-triazol-4-yl]imidazo[1,2-a]pyrazine

The title compound was prepared in a similar manner as described for Example 9, Step 3 from 1-[1-(3,4,5-trichlorobenzyl)-1H-1,2,3-triazol-4-yl]ethanone and pyrazin-2-amine.

¹H NMR (500 MHz, acetone-d₆): δ 8.96 (s, 1H), 8.57 (s, 2H), 8.45 (s, 1H), 7.89-7.92 (m, 1H), 7.69 (s, 2H), 5.82 (s, 2H). MS: m/z 379, 381 (MH⁺).

### Example 11

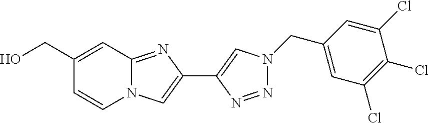

### {2-[1-(3,4,5-Trichlorobenzyl)-1H-1,2,3-triazol-4-yl]imidazo[1,2-a]pyridin-7-yl}methanol

The title compound was prepared in a similar manner as described for Example 9, Step 3 from 1-[1-(3,4,5-trichlorobenzyl)-1H-1,2,3-triazol-4-yl]ethanone and (2-aminopyridin-4-yl)methanol.

¹H NMR (500 MHz, acetone-d₆): δ 8.47 (d, 1H), 8.04 (s, 1H), 8.22 (s, 1H), 7.62 (s, 2 H), 7.47 (s, 1H), 6.89 (d, 1H), 5.78 (s, 2H), 4.70 (s, 2H). MS: m/z 408, 410 (MH⁺).

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 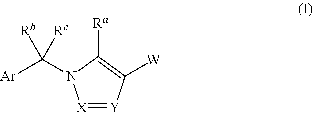
or a pharmaceutically acceptable salt thereof; wherein
X and Y are each independently CH or N;
W is heteroaryl selected from the group consisting of: 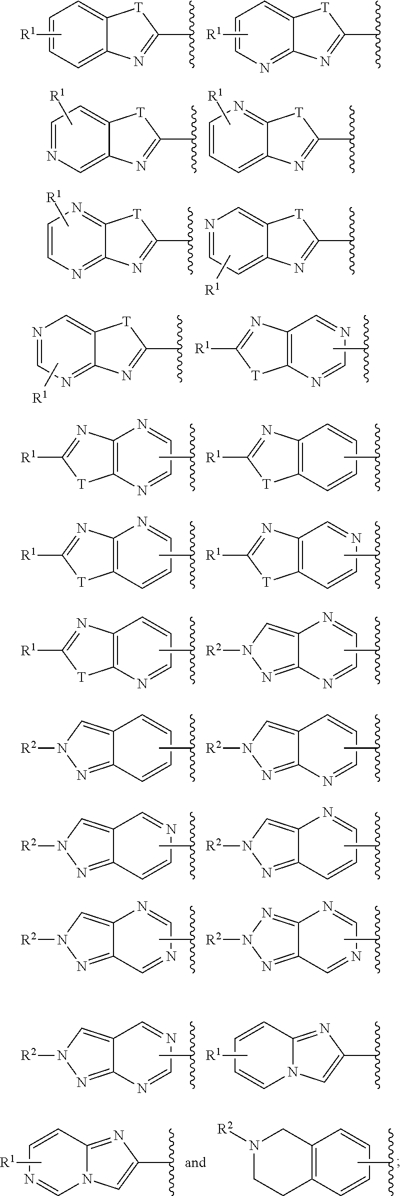
wherein W is further optionally substituted with one to two substituents independently selected from R⁴;
R¹ is selected from the group consisting of:
—(CH₂)ₚCO₂H,
—(CH₂)ₚCO₂C₁₋₄ alkyl,
—Z(CH₂)ₘCO₂H,
—Z(CH₂)ₘCO₂C₁₋₄ alkyl,
—(CH₂)ₙOR⁶,
—(CH₂)ₙ—CONR⁶R⁷,
—(CH₂)ₙ—OCONR⁶R⁷,
—(CH₂)ₙ—SO₂NR⁶R⁷,
—(CH₂)ₙ—SO₂R⁸,
—(CH₂)ₙ—NR⁹SO₂R⁸,
—(CH₂)ₙ—NR⁹CONR⁶R⁷,
—(CH₂)ₙ—NR⁹COR⁹, and
—(CH₂)ₙ—NR⁹CO₂R⁸;
R² is —(CH₂)ₘCO₂H or —(CH₂)ₘCO₂C₁₋₃ alkyl;
each m is independently an integer from 1 to 3;
each n is independently an integer from 0 to 3;
each p is independently an integer from 0 to 3;
T is O, S, or NR⁵;
Z is O, S, or NR⁵;
each R⁴ is independently selected from the group consisting of:
hydrogen,
halogen,
cyano,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl,
carboxy,
C₁₋₄ alkyloxycarbonyl, and
C₁₋₄ alkylcarbonyl;
R⁵ is hydrogen or C₁₋₄ alkyl wherein alkyl is optionally substituted with one to five fluorines;
R⁶ and R⁷ are each independently selected from the group consisting of
hydrogen,
(CH₂)ₙ-phenyl,
(CH₂)ₙ—C₃₋₆ cycloalkyl, and
C₁₋₆ alkyl, wherein alkyl is optionally substituted with one to five substituents independently selected from fluorine and hydroxy and wherein phenyl and cycloalkyl are optionally substituted with one to five substituents independently selected from halogen, hydroxy, C₁₋₆ alkyl, and C₁₋₆ alkoxy, wherein alkyl and alkoxy are optionally substituted with one to five fluorines;
or R⁶ and R⁷ together with the nitrogen atom to which they are attached form a heterocyclic ring selected from azetidine, pyrrolidine, piperidine, piperazine, and morpholine wherein said heterocyclic ring is optionally substituted with one to three substituents independently selected from halogen, hydroxy, C₁₋₆ alkyl, and C₁₋₆ alkoxy, wherein alkyl and alkoxy are optionally substituted with one to five fluorines;
each R⁸ is independently C₁₋₆ alkyl, wherein alkyl is optionally substituted with one to five substituents independently selected from fluorine and hydroxyl;
R⁹ is hydrogen or R⁸;
Ar is phenyl or pyridyl each of which is optionally substituted with one to five substituents independently selected from the group consisting of:
halogen,
C₁₋₆ alkyl optionally substituted with one to five fluorines,
C₂₋₆ alkenyl,
C₂₋₆ alkynyl,
C₁₋₆ alkylthio, optionally substituted with one to five fluorines,
C₁₋₆ alkoxy, optionally substituted with one to five fluorines, and
C₃₋₆ cycloalkyl;
R^{a} is hydrogen or C₁₋₄ alkyl wherein alkyl is optionally substituted with one to five fluorines; and
R^{b} and R^{c} are each independently hydrogen, fluorine, or C₁₋₄ alkyl wherein alkyl is optionally substituted with one to five fluorines;
or R^{b} and R^{c} are taken together to form a 3- to 6-membered saturated carbocyclic ring optionally containing a heteroatom selected from the group consisting of O, S, and N.

2. The compound of claim 1 wherein X and Y are both N.

3. The compound of claim 2 wherein R^{a}, R^{b}, and R^{c} are each hydrogen, and Ar is phenyl substituted with one to five substituents independently selected from the group consisting of halogen and C₁₋₄ alkyl.

4. The compound of claim 1 wherein W is heteroaryl selected from the group consisting of:

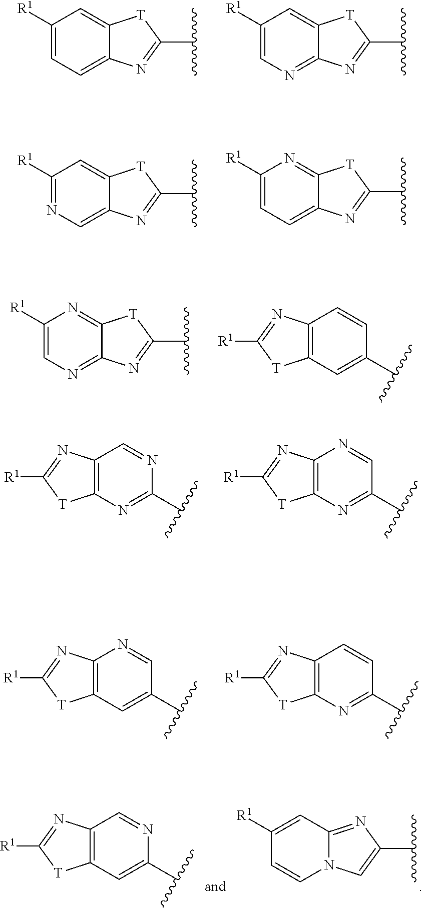

5. The compound of claim 4 wherein T is O or S.

6. The compound of claim 5 wherein T is S.

7. The compound of claim 4 wherein X and Y are both N.

8. The compound of claim 7 wherein R^{a}, R^{b}, and R^{c} are each hydrogen, and Ar is phenyl substituted with one to five substituents independently selected from the group consisting of halogen and C₁₋₄ alkyl.

9. The compound of claim 1 wherein W is heteroaryl selected from the group consisting of:

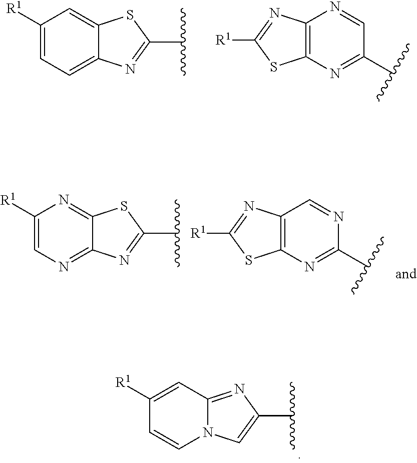

10. The compound of claim 9 wherein X and Y are both N.

11. The compound of claim 10 wherein R^{a}, R^{b}, and R^{c} are each hydrogen, and Ar is phenyl substituted with one to five substituents independently selected from the group consisting of halogen and C₁₋₄ alkyl.

12. A pharmaceutical composition comprising a compound in accordance with claim 1 in combination with a pharmaceutically acceptable carrier.

13-17. (canceled)

18. A compound selected from the group consisting of 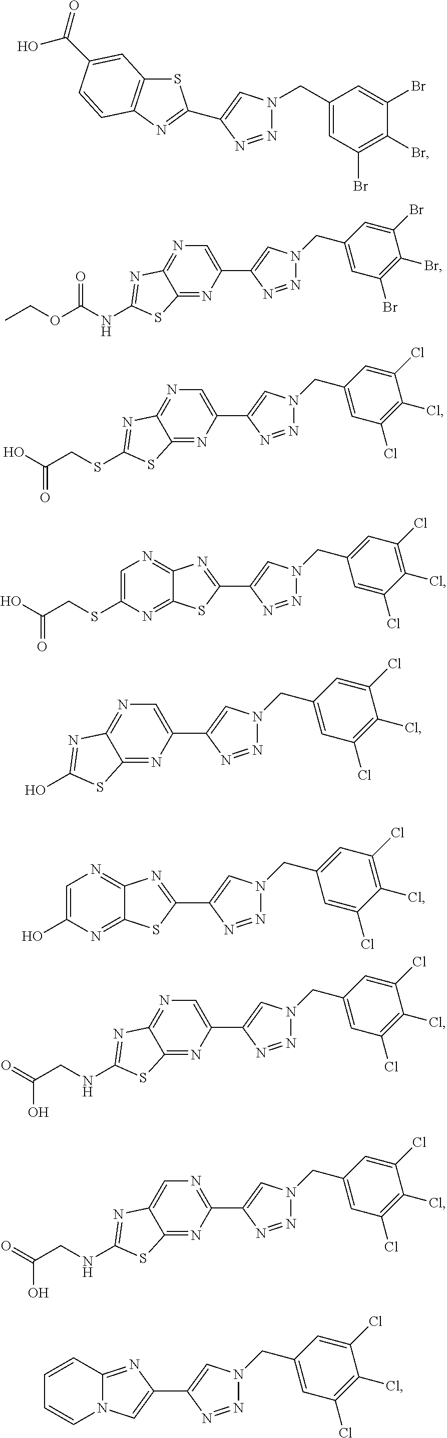 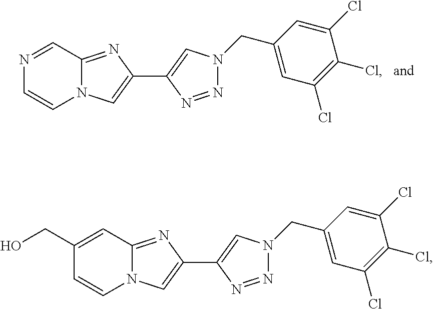
or a pharmaceutically acceptable salt thereof.

19. A method of treating hyperglycemia, diabetes or insulin resistance in a mammal in need thereof which comprises the administration to the mammal of a therapeutically effective amount of a compound of claim 1.

20. A method of treating a lipid disorder selected from the group consisting of dyslipidemia, hyperlipidemia, hypertriglyceridemia, hypercholesterolemia, low HDL, and high LDL in a mammal in need thereof which comprises the administration to the mammal of a therapeutically effective amount of a compound of claim 1.
